(19) United States
(12) Patent Application Publication
(10) Pub. No.: US 2018/0242858 A1
(43) Pub. Date: Aug. 30, 2018

(54) Method of registering the intervals between adjacent R-peaks of the ECG signal with the one hand in order to diagnose and assess the state of the human body and Heart Rate Variability wearable monitoring device

(71) Applicants: Tetiana BOTSVA, Dnipro (GB); Dmytro CHERNETCHENKO, Dnipro (GB)

(72) Inventors: Tetiana BOTSVA, Dnipro (GB); Dmytro CHERNETCHENKO, Dnipro (GB)

(21) Appl. No.: 15/442,631

(22) Filed: Feb. 25, 2017

(51) Int. Cl. A61B 5/0245; A61B 5/00; A61B 5/024; A61B 5/0402; A61B 5/0456; A61B 5/046; A61B 5/0468

(52) U.S. Cl. CPC A61B 5/0245; A61B 5/0006; A61B 5/02405; A61B 5/0402; A61B 5/0408; A61B 5/046; A61B 5/0468; A61B 5/7285; A61B 5/024; A61B 5/0456

(57) ABSTRACT

The invention relates to a method, system and program product to obtain R-R-intervals. The described herein wearable device is intended for recording the electrical signal caused by the work of the heart. The signal which is a potential difference is recorded from the one hand, by two dry electrodes relative “reference” electrode. This method allows to record the signal in motion. The accuracy of the R-R-intervals values calculated by using this method is sufficient for HRV analysis. The method comprises the following steps: registration of the ECG signal, amplification, filtering, AD-conversion, R-peak detection and the calculation of R-R-intervals. The resulting array of data is stored in device Flash memory and transmitted via wireless communication to a mobile device. Using the application installed on the mobile device and the R-R-intervals array HRV analysis is performed for example to analyze and assess the emotional state of the user.

### BACKGROUND OF THE INVENTION

### Field of the Invention

The present invention, which include method, system and software, is in the field of monitoring cardiac system of a human in purpose of diagnostics of the human condition in everyday life, during exercising and for self-control as well as medical purposes. Collected data by the described herein invention can be used for Heart Rate Variability (HRV) analysis.

### Description of the Related Art

Increasing the diversity of personal health monitoring devices available on the market is caused by growing consumer interest in personal health. Almost all of these devices recorded heart rate and majority or them use photo sensor for this purpose. The one common for wearable devices photoplethysmography method does not meet the accuracy requirements for HRV analysis, which requires value of RR-intervals, that is time interval between two successive heartbeat. Required accuracy can be achieved only by using ECG recording. Information obtained as a result of processing a single-lead ECG may be sufficient to determine the functional state of human (e.g. heart rate, key information about cardiac disease and even emotional state).

There are several known devices that register electrocardiogram. The one of them, The Kardia Band is described as follows on product resources (“Kardia™ Band by AliveCor® Instructions For Use” https:www.alivecor.com/user-manuals/kardia-band-instructions-for-use-en.pdf) The Kardia Band for Apple Watch will function in a similar way, with a small, integrated metal sensor in the band communicating with the company's new app to take wrist-worn ECG readings. Record and analyses of 30-second a single-lead ECG could be done by simply place thumb of one hand on Kardia Band's integrated sensor on the other hand that communicates with the Apple Watch—compatible Kardia app. The Atrial Fibrillation (AF) Detector then uses Kardia's automated analysis process (algorithm) to instantly detect the presence of AF in an ECG, the most common cardiac arrhythmia and a leading cause of stroke.

Also included is the Normal Detector, which indicates whether your heart rate and rhythm are normal, and the Unreadable Detector, which tells when to retake an EGG so physicians receive only the highest quality recordings. The collected recordings are stored and can be viewed in the iPhone version of the Kardia app, and even sent to a patient's preferred doctor if any suspicious activity is gathered during a reading. App can also connect to Apple's stock Health app, so users can integrate their ECG readings into established fitness data like step count and calorie intake to further bolster an overall assessment of their well-being. Medical Grade: the first medical grade electrocardiogram (ECG) band for the Apple Watch and a breakthrough in proactive heart health. Instant ECG Analysis: With a quick, discreet touch of the Watch band, know instantly if heart rhythm is normal or if atrial fibrillation (AF) is detected in ECG. Track and Share: Capture reliable heart activity data and relay it to doctor to inform diagnosis and treatment plan. More Than ECGs: Use voice memos to keep track of palpitations, shortness of breath, dietary habits and exercise patterns. Integrate with Health app and Google Fit for personal heart health insights. One-time setup and activation.

### SUMMARY OF THE INVENTION

An object of the present invention is to provide a method or recording the signal of electrical activity of the heart for subsequent isolation R-R-intervals and device for its implementation. The objects of the invention are achieved by a method and an electronic device that are characterized by what is stated in the Claims.

According to an aspect of the invention, there is provided a method of recording the ECG signal which is a sequence of values of the potential difference (voltage) caused by the heart's electrical activity. The most high-amplitude peaks of the signal are R-peaks which correspond to the time of the heart muscle reduction. The present herein method provides the required accuracy for the registration of R-R-intervals. A distinctive feature of this method is the possibility of registration the signal with one hand (unlike Kardia Band described above) using dry electrodes.

There is provided a method comprising: registration of the signal electrical activity of the heart; measured signal amplification, digitization and transfer for further processing. During subsequent processing R-R-intervals are isolated as msec intervals between R-peaks—the most high-amplitude elements allocated in the recorded signal. Also, from the recorded signal is calculated heart rate value. Calculated values of R-R-intervals is further used for HRV analysis.

According to another aspect of the invention, there is provided an electronic device comprising: sensor unit taking down an electrical signal from the skin and consisting of two active electrodes and a bias electrode; analog filtering and signal amplification unit comprising an automatic gain control system, which changes the gain depending on the level of the recording signal; microcontroller unit where the signal is digitized and subjected to further processing, during which the R-peak detection and the calculation of R-R-intervals take place. The electronic device further comprises: a memory unit for storing the received data; wireless communication module for data transmission to a mobile device for postprocessing and analysis of the data and visualizing the result; miniature battery and power management and charge control unit.

As a third aspect of the invention, the invention provides an algorithm of the described here electronic device that permit recording ECG signals by dry electrodes from a limited portion of the user's hand with sufficient quality for further processing, as well as perform the processing itself, recording and data transmission. To achieve the required accuracy of the recorded data algorithm comprises the following steps: check the quality of the electrodes contact to the human body by measuring impedance; control signal recording length no less than a certain predetermined time (130 seconds); check the signal level and adjustment of the corresponding gain. At the end of the recording of the original signal, the process proceeds to the filtration stages of the recorded signal, detecting subsequent R-peaks, calculate R-R-intervals values and recording them into the memory and transmitting to the mobile device via wireless communication.

As a fourth aspect of the invention, the method and apparatus described herein provide R-peaks isolation function of the ECG signal from the electrical signal recorded from user's hand. Execution of this function is provided by the filters which coefficients are specially configured to suppress noise and signals superimposed on the ECG signal (e.g., such as myogram signal) and by the work of peak-detector which selects only R-peaks from the ECG signal. This feature allows accurate to several (up to 4) milliseconds to determine the position of the R-wave in the timeline that is much exceeds the value of accuracy often used for this purpose photoplethysmography method mentioned above.

The method and electronic device of the invention provide several advantages. Recording the ECG signal performed with one hand by means of a small electronic device placed on the user's wrist that meets the requirements of wearable devices and provides freedom of user's movements. The quality of the recorded signal and the accuracy of the computed values of RR-intervals are sufficient to calculate the heart rate and further HRV analysis. Wireless communication between the electronic device and a mobile device provides further processing, storage and visualization of the collected data.

### BRIEF DESCRIPTION OF THE DRAWINGS

The appended drawings, in which there is shown one of the possible realisation (embodiment) of the present invention are provided in purpose of better understanding of the following detailed description of the invention. It should be remembered that the various embodiments of the present invention are not limited to the precise arrangements and instrumentalities shown in the drawings and specific values of the parameters should be considered only as an illustration of a particular example implementation.

In the Drawings:

FIG. 1a top view of the location the electronic device on the user's hand;

FIG. 1b bottom view of the location the electronic device on the user's hand;

FIG. 2a shows the placement of the information indicating means on the top of device housing;

FIG. 2b shows-the position of the electrodes on the bottom of device housing;

FIG. 3 is cross-section of the electrode shape and location in the housing;

FIG. 4 is block diagram of the electronic device

FIG. 5 shows the algorithm of the embedded software

FIG. 6 is algorithm of Mobile App

FIG. 7 shows the algorithm of R-peaks detector

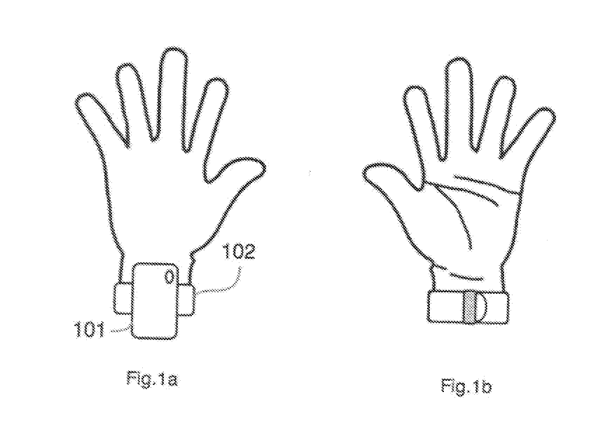

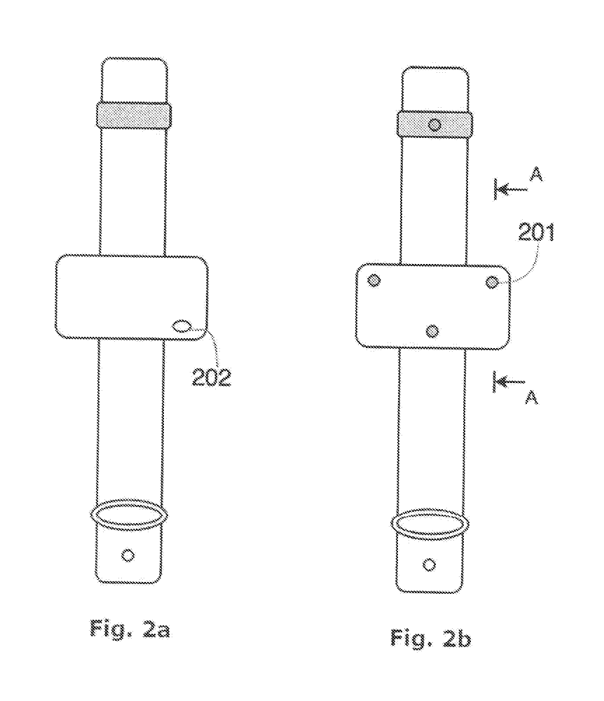

### DETAILED DESCRIPTION OF THE PREFERRED EMBODIMENT

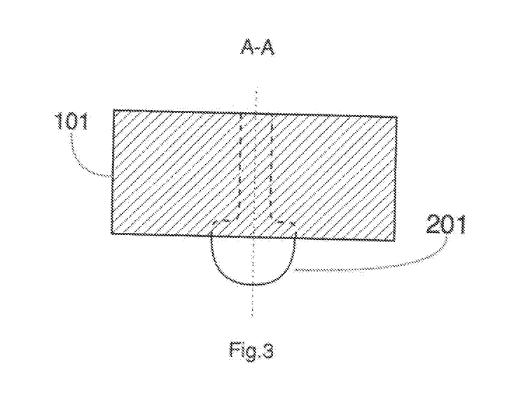

The electronic device described in the present invention is placed in the housing 101 and fixed to the users hand by the strap 102. The electrodes 201 are “dry” (which means it doesn't require special gel or water) and are located on the external bottom surface of the housing in direct contact with the human skin, their pressing to skin is provided by means of the device housing strap 102. Also there are indication means 202 on the external top surface of the housing. Electrodes have rounded shape 201 and ace placed in the wall 101 of the housing as shown on FIG. 3. Advantages of a rounded shape (convex shape of the fungus) electrodes; ensure good contact with the skin (flat electrodes do not provide a good snuggling and contact can be interrupted while moving a hand or housing in relation to the hand; the spherical shape of the electrodes may be adversely affected on the useful signal associated with friction on the skin); user comfort—electrodes provide good contact without digging into the skin, thereby not causing discomfort during prolonged wearing device.

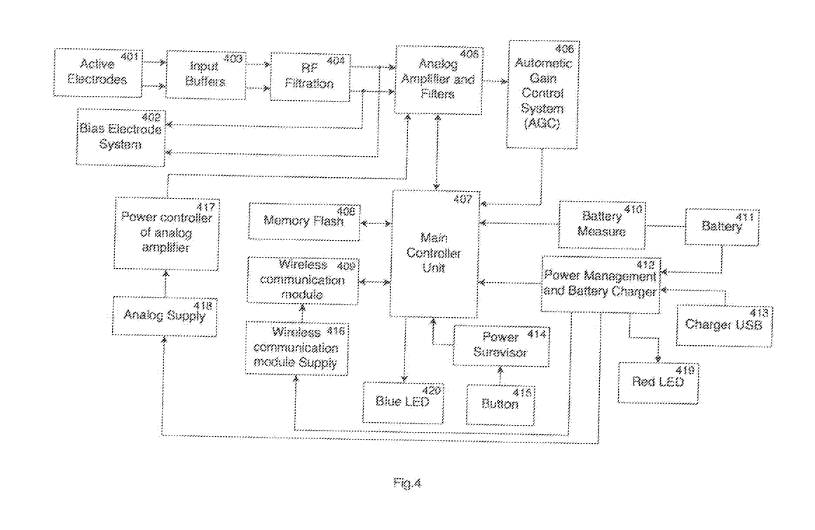

The electrodes record the minor fluctuations of the electric field generated by the human heart with an amplitude of about 0.5-1.5 mV. The potential from active electrodes 401 (look FIG. 4) is transmitted through a cascade of buffer amplifiers 403 and RFI 404 to a differential instrumentation amplifier analog signal 405, that executes amplification potential difference between the two active electrodes 401, subsequent block 405 of the tract performs the primary filtering and noise reduction of the received signal, and sets the working frequency domain of the recorded signal in the range from 0.5 to 100 Hz. Fixed gain of the path is 100.

Full analog path gain factor is adjusted by the AGC (automatic gain control) with digital control 406. The gain of the programmable amplifier can assume the values 1, 2, 4, 10, 16, 32. As a result, the minimum gain of the analog tract is 100, and the maximum is 3200.

To set the amplifier linearity, further suppress phase noise and dynamic range of potentials applied the relative “reference” electrode technology, implemented in block 402.

After initial processing signal enters the a microcontroller unit 407, to the input of analog-to-digital converter (ADC) for digitizing the signal, and further processing and transmission to the mobile device via wireless communication 409.

The analog signal power amplifier is controlled by a circuit 417 formed on the transistor switch.

Power of the device is provided by an external lithium polymer battery 411. The battery is connected to the device PCB.

The stabilized voltage for all components is provided by two voltage formers: first is analog supply block 418 for power whole analog past of the device, and the other for the wireless communication unit power supply 416. The value of the stabilized voltage is Ustab=3.3V.

To check the battery charge is used microcontroller unit 407, as well as a chain of batteries measuring 410 operating on a “divider”.

To charge the battery from an external USB-adapter (5V) using charge unit 412 which is connected with USB connecter 413 built-on the device PCB. Battery charge unit is equipped with an LED 419 to indicate battery charge process, which turns off at the end of the charging process.

The microcontroller unit 407 sends data to the wireless communication-module 409, for further transmission over the wireless link to a destination device (mobile phone, a tablet, etc. devices). Microcontroller unit 407 performs the function of digital processing of the data using the following methods: mathematical averaging techniques; filtration using finite impulse response (FIR); the method of discrete Fourier transform (DFT). After digital processing the input signal sample, there is detection of ECG R-peaks for the subsequent computation the distance between R-peaks so-called RR-intervals. The result of the R-peaks detector is an array of RR-intervals.

The microcontroller 407 in real time measurement of the sample data of ECG performs control algorithm of automatic gain control (AGC) of the analog path. AGC occurs on detection algorithm oi peaks at the frequencies corresponding to R-peaks in the measured signal and comparing foe amplitude of the detected peaks to a threshold amplitude of the minimum necessary for optimal detection of ECG peaks. If the average amplitude of the detected peak exceeds the threshold (locking analog signal) the gain value is decreased by one position (taking into account the standard number of available gain 1, 2, 4, 5, 8, 10, 16, 32), otherwise—gain increases tip one position.

Obtained array of RR-intervals is stored in Flash-Memory 408. For data storage Bash memory is used with a parallel interface and a size from 4 MB. This allows the device to operate in standalone (autonomous) mode without synchronization with the mobile device tip to 48 hours. When synchronization the device with a mobile device is happen on a schedule or on-demand measured data is read and the subsequent transfer of the new data files to a mobile device. During transferring data package from the device to the mobile device provides checking of the control CRC sum for each measurement to improve reliability of the transmitted data. If the fie was not sent to or sent to the mobile device with an error for some reason, there is a re-sending the packet takes place.

Power supervisor 414 is a tracking microcontroller and performs the following functions: power management of the wireless communication unit; initial turn on/off device, with a single long press button 415; awakening device with a single short press of a button; the insertion device hi firmware update mode (by double-clicking the button); Reset the device parameters when the button is pressed three-fold, respectively. Processes of turn on and reset (after a one-click button) is indicated by a blue LED 420 that lights up three times with an interval of 0.5 sec.

Recording of the 130 seconds sample is automatically every half an boor for example, and when starting measuring by the user in on-demand mode. At the end of the measurement, the device is immersed in a deep sleep to minimize the energy consumption until the next scheduled measurement. In the case of single short press of the button 415, it is possible to perform measurement on demand by running a process from a mobile device.

With the help of a mathematical apparatus HRV analysis is made and determined the emotional state (eg., the stress index), physiological state (eg., the balance of the nervous system). The user is shown the instantaneous diagram of the state and generalized diagram per day. The device and method described herein provide an opportunity to improve the HRV analysis, expanding it by the range of tools for assessment of sports readiness, as well as the medical diagnosis—such as risk of acute cardiovascular events, etc.

Getting and interpreting the signal from the device. Receives a signal as a series of values of the potential difference caused by the heart's electrical activity. Then, through foe developed mathematical apparatus, R-peaks are determined and calculated R-R-intervals in ms. This sequence (array) of the R-R-intervals is a valuable and informative results of measurements. By itself, the received signal (raw data) is not interpreted and is not transmitted to external applications. The array of R-R-intervals is a source of information on heart rate variability (HRV). Mathematical processing of the array to produce a geometric and frequency characteristics (eg, SDDN, RMS-SD, TINN, LF/HF et al.) and interpretation of the calculated values on the basis of relevant clinical studies, standards and scientific publications may be made in the application on the mobile device or in the cloud web service. The application can be both development PLANEXTA, and third-party developers using PLANEXTA Sence Hub API.

The device user can get, depending on the scenario of using the device and additional software products, the following information: display R-peaks corresponding to the heartbeat; mathematical characteristics of the array R-R-intervals (known and conventional widely described in the literature); diagnostic assumptions on the basis of the above-mentioned characteristics of the psycho-emotional state, the risk of acute cardiovascular events, quality of sleep, etc.

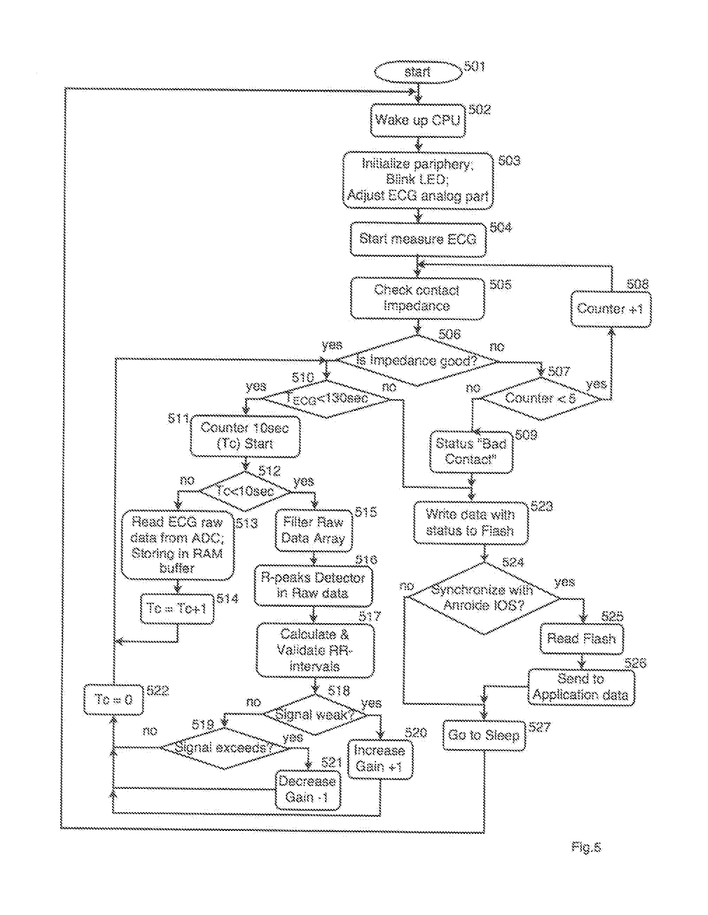

The algorithm of the microcontroller is shown on FIG. 5.

At the time of the supply voltage to the device electrical circuit occurs start 501 and execution of the main program code stored in nonvolatile FLASH-memory of the microcontroller (MCU).

Microcontroller or microcontroller unit (MCU) is used to control peripherals, modes electronic device, communication with the external mobile device, as well as the registration and send data (RR-intervals). In the first stage main microcontroller initializes peripheral 503, initializes input-output posts data (GPIO), sets the analog to digital converter (ADC), digital-to-analog converter (DAC), the driver direct memory access (DMA), and initializes the hardware timers (TIM2, TIM3, TIM4, TIM9).

Upon successful completion of the first phase started starting LED-indication 503—3 short blinks red bi-color LED, duration T=1 sec. and the intervals between blinks Td=0.5 s.

Microcontroller (MCU) checks the connection with the device via wireless communication, and sets it in standby receive mode control commands from an external paired device.

The device can operate in two basic modes—“active mode” (the bracelet is worn on the wrist), and “battery charging mode” (the bracelet is connected using the USB-cable to an external power source). In case when the USB-cable is connected—the microcontroller (MCU) is in standby mode, there is a battery charge.

In the case when USB-cable is disconnected from the device, a microcontroller (MCU) switches to the operating mode: measuring two times per hour by schedule, and waiting for the next measurement (SLEEP MODE).

At the start of the scheduled measurement 504.

First step is analysis of the quality of the contact electrodes to skin (impedance cheek) 505, 506 in pulsed mode: registers the displacement of the electrode potential by skin stimulation through electrodes during 800 ms. If the measured value is below the threshold value is re-measurement of electrode impedance at intervals of 5 seconds. If the impedance was below the threshold of more than 5 times 507, 508, the measurement ends with an error status “Bad impedance” 509 creates a corresponding entry in the Flash memory 523.

If the impedance is above a threshold, the program proceeds to register new data from the ADC within sampling rate of 500 Hz (T=0.002 seconds) during 130 seconds 510. The received data 513 is written to the circular buffer 511, 512, 514 which size corresponds to a time window (for example 10 sec). When the buffer has filled the data is processed by a digital filter 515, and transmitted to the peak detector 516 for detecting and validation of RR intervals 517.

If the detected peak amplitude less than the threshold 518—the gain of the AGC is incremented by one position 520, otherwise—AGC gain is reduced by one position 521.

After the 130 second measurement, the resulting array of RR-intervals stored in Flash-memory 523. Checking if the connection with the mobile device is available 524. If the device is available to synchronize the new record in the background 525, 526, otherwise—the bracelet goes to sleep 527 until the next scheduled measurement.

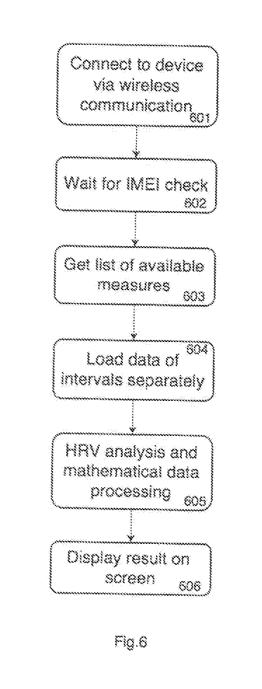

Algorithm SenceHub mobile application is shown on FIG. 6:

A connection 601 is established between the mobile and electronic devices (or band) via wireless communication, the data packet with IMEI initially sent to the band. Expected to check 602 IMEI mobile device and IMEI values recorded in the internal non-volatile memory of the electronic device. If the values are the same—that allowed further interaction between paired devices, if not—the connection is terminated at the initiative of the band. If the IMEI value in the band memory is empty—application invites you to initiate a pair with the device.

The application sends a request 603 to a bracelet to get a list of all available measurements in memory.

When mobile application and device synchronizing a new array of RR-intervals from the device memory is transferred to the mobile device via wireless communication 604. During transmitting data packets used method of comparing the checksums of the data on side of the electronic device and the mobile device. If the packet transmission was unsuccessful, or the data does not match, the mobile device initiates a retransmission of data.

Developed HRV math for processing RR-interval 605 is used to process all data and the calculation of physiological parameters (stress, vitality, calculating the emotional state)

Data is written to the Database applications and visualized 606 the mobile device screen.

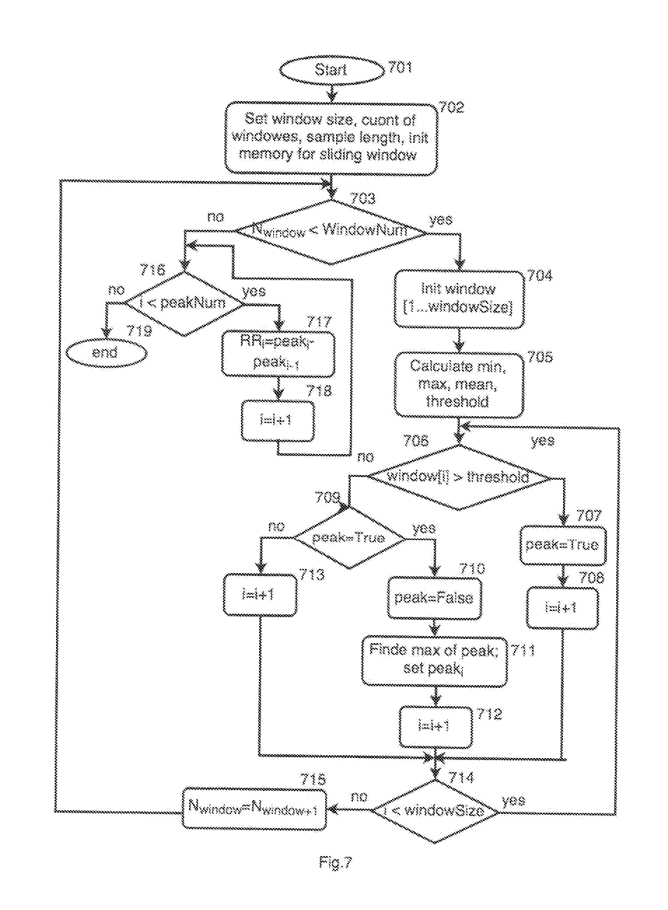

R-peaks Detector Algorithm shown on FIG. 7.

The process of detecting the R-peaks and calculate R-R intervals comprising following step.

Calculate the size of the sliding window, the number of partitions of the signal sample, initialize a memory for buffer of the sliding window 701, 702.

Runs window counter which determines the number of the current window, and varies from 1 to the number of partitions of the sample 703, 704.

For the current data in the buffer of the sliding window is calculated statistical parameters, such as maximum, minimum, mean, and the computed value of the dynamic threshold signal level within this window 705.

For each signal value in the window buffer checks exceeding threshold values of amplitude. If the threshold has been exceeded 706, the set value of the peak detection flag signal fragment (peak flag) 707. If the signal is passed below a threshold level 706, and peak flag has been previously installed 709 is registered by the end of the peak, and the peak flag is cleared 710, find the index corresponding to the maximum value of the signal amplitude for peak track 711. The index of the founded maximum peak is recorded in a global array of peaks 714, 715.

After the exhaustive search of all window intervals 703,716, calculate the values of R-R intervals, as the difference between indexes of adjacent peaks, knowing the frequency of sampling 717, the values are translated into time units of measurement (ms) and is recorded in a global array of R-R intervals, for further work with them 719.

While specific embodiments have been described in detail in the foregoing detailed description and illustrated in the accompanying drawings, it will be appreciated by those skilled in the art that various modifications and alternatives to those details could be developed in light of the overall teachings of the disclosure and the broad inventive concepts thereof. It is understood, therefore, that the scope of the present invention is not limited to the particular examples and implementations disclosed herein, but is intended to cover modifications within the spirit and scope thereof as defined by the appended claims and any and all equivalents thereof.

1. A method of diagnosing and assessing a body condition (state) by a recording device, comprising a housing with an element for its fixation on a user's hand for a necessary electrode contact with a user's skin, active electrodes and a reference electrode placed on a bottom surface of the housing; a microprocessor with a memory, a power supply, a wireless communication unit are placed inside the housing; wherein contact electrodes, the power supply, the wireless communication unit have data communication with the microprocessor,
the method comprises the steps of:
initiating a search of a registering devices (by scanning via wireless communication) and establish a wireless connection of an external electronic device (an external mobile device) with the recording device via the wireless communication unit
calling (demand) function of measuring impedance of the skin and, after a comparison with a reference data, interpretation is returned in forms of “good/bad contact with the skin”
calling function of receiving ECG signal of a certain duration
retaining a sample (array of numbers—instant potential difference values)
performing search of points satisfying R-peaks features (highest amplitude ECG signal peaks, further R-peaks)
retaining an array R-peaks as a timestamps corresponding to the time of the peak
calculating the difference: R-R-intervals (intervals between R-peaks), appropriate for processing and interpretation by standard certified procedures and save them in the memory,
transmitting the values of RR-intervals via the wireless communication unit to the external electronic (mobile) device
perform Heart Rate Variability (further HRV) analysis at the external electronic device and visualize measurement results.

2. A device for diagnosis and evaluation of body condition (R-R-intervals detector, Heart Rate Variability wearable monitoring device) comprising
a housing with top and bottom surfaces,
contact electrodes placed on the bottom surface of the housing,
electronic circuit realised on PCB board placed inside the housing and include a microprocessor, a power supply, a wireless communication unit,
and an element for fixing the housing on a user's hand for a necessary contact of the contact electrodes with a user's skin, wherein power supply and wireless communication unit have data communication with the microprocessor

3. The device for diagnosis of claim 2 wherein the contact electrodes comprising at least two measuring active electrodes and at least one reference electrode.

4. The device for diagnosis of claim 2 wherein the electronic circuit comprising
an analog signal amplifier;
a microcontroller unit (MCU);
an analog amplifier power control circuit (Pre-amp voltage control);
a digital circuit voltage regulator (Voltage regulator wireless communication unit);
a voltage regulator of analog circuit;
a charging circuit and power supply control unit (Battery charger);
a wireless communication unit power control;
a battery voltage meter;

5. The device for diagnosis of claim 4 further comprising an electrode contact quality measurement module

6. The device for diagnosis of claim 2 wherein the element for fixing the housing on the user's hand is in the form of a strap

7. The device for diagnosis of claim 2 wherein the contact electrodes comprising at least one reference electrode

8. The device for diagnosis of claim 3 wherein the microprocessor comprising a reference electrode circuit (Reference electrode preamp);

9. The device for diagnosis of claim 2 wherein information display means are placed on the top surface of the housing.

10. A R-R intervals registration method for diagnosis and assessment of a body state, using a device comprising a housing with an element for fixation the housing on an user's hand for necessary electrodes contact with an user's skin,
dry active electrodes and a dry reference electrode is placed on a bottom surface of the housing;
located within the housing a microprocessor with a memory, a power supply, a wireless communication unit, wherein the microprocessor processes data received from the wireless communication unit,
the method comprising
recording a signal from a restricted area of user's skin on hand using the dry electrodes,
recording the signal as a series of potential difference values caused by an electrical activity of the heart,
the measured signal is amplified, digitized isolated R-R-intervals as the intervals in ms between R-peaks—the most high-amplitude elements allocated by a recorded device,
calculating R-R-intervals are transmitted to an external electronic device for further processing using the wireless communication unit at the external electronic device according to the calculated R-R-intervals to determine emotional state (eg., the stress index), physiological state (eg., the balance of the nervous system).

11. The method for obtaining R-R-intervals of claim 10 wherein for recording the signal are used at least two dry electrodes, and a recorded signal is differential signal from at least two main electrodes relative to the at least one reference electrode.

12. The method for obtaining R-R-intervals of claim 10 wherein registration takes place automatically, for example, every hour.

13. The method for obtaining R-R-intervals of claim 10 wherein fluctuations of the recorded electric field generated by the human heart have an amplitude of about 0.5-1.5 mV.

14. The method for obtaining R-R-intervals of claim 10 wherein the “dry” electrodes are in direct contact with the human skin and pressed to the skin by the device housing fixing element (housing strap).

15. The method for obtaining R-R-intervals of claim 10 wherein the recording device measures the battery level and indicates the status of the device using the light indication.

16. The method for obtaining R-R-intervals of claim 10 wherein registration occurs by a targeted request from the user (on-demand mode).

17. The method for obtaining R-R-intervals of claim 14 wherein the electrode contact quality checked by impedance between the skin and the electrodes.

18. The method for obtaining R-R-intervals of claim 10 wherein the assessment of emotional state made by the heart rate variability analysis (HRV).

19. The method for obtaining R-R-intervals of claim 10 wherein measurements are performed on the wrist, and the electrode spacing is at least 30 mm.
